(19) United States
(12) Patent Application Publication
(10) Pub. No.: US 2024/0044756 A1
(43) Pub. Date: Feb. 8, 2024

(54) LOW-COST ENVIRONMENTAL CONTAMINANT ANALYSIS FOR CUSTOMIZED TREATMENT

(71) Applicant: EMED LABS, LLC, Miami, FL (US)

(72) Inventor: Nicholas Atkinson Kramer, Wilton Manors, FL (US)

(21) Appl. No.: 18/366,593

(22) Filed: Aug. 7, 2023

Related U.S. Application Data
(60) Provisional application No. 63/370,558, filed on Aug. 5, 2022.

(51) Int. Cl. G01N 1/34

(52) U.S. Cl. CPC G01N 1/34; G01N 2001/1056

(57) ABSTRACT

Systems, methods, and devices for identifying molds or bacteria contained in a sample are disclosed herein. In some embodiments, a device can be used to perform analysis of a sample and identifying molds or bacteria contained within. The device may include a sample collection tool for collecting and holding a sample. The device may also include a sample receptacle, which may have an agar portion disposed within the sample receptacle and an inlet port. There may be a binary tree of medicines distributed within the agar portion. The inlet port may be configured to receive an end of a sample collection tool, wherein full insertion of the sample collection tool through the inlet port causes the sample to be placed in contact with the agar portion. Mold or bacteria from the collected sample may grow or attempt to grow throughout the agar, through the binary tree, and visual analysis of the growth paths may allow the mold or bacteria to be identified and the best combination of medicines to be determined.

### CROSS-REFERENCE TO RELATED APPLICATIONS

Any and all applications for which a foreign or domestic priority claim is identified in the Application Data Sheet as filed with the present application are hereby incorporated by reference under 37 CFR 1.57.

This application claims the benefit of U.S. Provisional Patent Application No. 63/370558, entitled “ LOW-COST ENVIRONMENTAL CONTAMINANT ANALYSIS FOR CUSTOMIZED TREATMENT,” filed Aug. 5, 2022, the contents of which are incorporated by reference herein in their entirety.

### TECHNICAL FIELD

The embodiments of the disclosure generally relate to systems, methods, and devices for performing analysis of, or identifying contaminants in, a sample. More specifically, the embodiments relate to an at-home diagnostic test that can be used to identify molds or bacteria contained in a sample.

### BACKGROUND

Use of telehealth to deliver healthcare services has grown consistently over the last several decades and has experienced very rapid growth in the last several years. Telehealth can include the distribution of health-related services and information via electronic information and telecommunication technologies. Telehealth can allow for long distance patient and health provider contact, care, advice, reminders, education, intervention, monitoring, and remote admissions. Often, telehealth can involve the use of a user or patient's personal user device, such as a smartphone, tablet laptop, personal computer, or other device. For example, a user or patient can interact with a remotely located medical care provider using live video, audio, or text-based chat through the personal user device. Generally, such communication occurs over a network, such as a cellular or internet network.

Remote or at-home healthcare testing and diagnostics can solve or alleviate some problems associated with in-person testing. For example, health insurance may not be required, travel to a testing site is avoided, and tests can be completed at a testing user's convenience. However, remote or at-home testing introduces various additional logistical and technical issues, such as guaranteeing timely test delivery to a testing user, providing test delivery from a testing user to an appropriate lab, ensuring adequate user experience, ensuring proper sample collection, ensuring test verification and integrity, providing test result reporting to appropriate authorities and medical providers, and connecting testing users with medical providers who are needed to provide guidance and/or oversight of the testing procedures remotely.

### SUMMARY

For purposes of this summary, certain aspects, advantages, and novel features are described herein. It is to be understood that not necessarily all such advantages may be achieved in accordance with any particular embodiment. Thus, for example, those skilled in the art will recognize the disclosures herein may be embodied or carried out in a manner that achieves one or more advantages taught herein without necessarily achieving other advantages as may be taught or suggested herein.

All of the embodiments described herein are intended to be within the scope of the present disclosure. These and other embodiments will be readily apparent to those skilled in the art from the following detailed description, having reference to the attached figures. The invention is not intended to be limited to any particular disclosed embodiment or embodiments.

Environmental contaminants such as mold (i.e., mycotoxins) and bacteria can cause a wide variety of health issues that can be difficult to diagnose and even more difficult to treat. Even when a diagnosis is made (e.g., a mold contaminant diagnosis), the species is guessed or assumed, and highly nonspecific antifungals are given to the patient. These generic and overly aggressive antifungals can cause significant damage to a patient's gut and liver. Similar problems are common when diagnosing and treating bacterial infections.

A one-time-use, disposable, affordable cartridge system for environmental sample collection and testing that overcomes these problems is herein disclosed.

In some aspects, the techniques described herein relate to a device for performing analysis of a sample, the device including: a sample collection tool configured to collect and hold a sample; and a sample receptacle including: an agar portion, wherein the agar portion includes a binary tree of medicines distributed within the agar portion; and an inlet port configured to receive an end of a sample collection tool, wherein sufficient insertion of the sample collection tool through the inlet port causes the sample to be placed in contact with the agar portion, and wherein sufficient insertion of the sample collection tool through the inlet port causes the inlet port to be sealed thereby preventing outside contaminants from entering the sample receptacle.

In some aspects, the techniques described herein relate to a device, wherein the agar portion includes a recessed sample insertion area for receiving the sample collection tool when the sample collection tool is sufficiently inserted through the inlet port.

In some aspects, the techniques described herein relate to a device, wherein the agar portion further includes a recessed sample insertion area for receiving the sample collection tool when the sample collection tool is sufficiently inserted through the inlet port, wherein a seal covers the recessed sample insertion area, and wherein the seal is broken when the sample collection tool is sufficiently inserted through the inlet port thereby placing the sample in contact with the agar portion.

In some aspects, the techniques described herein relate to a device, wherein the sample collection tool includes a tube, swab, or strip.

In some aspects, the techniques described herein relate to a device, wherein the binary tree of medicines distributed within the agar portion includes a plurality of adjacent regions within the agar portion containing one or more antifungals or antibacterials.

In some aspects, the techniques described herein relate to a device, wherein the agar portion further includes a recessed sample insertion area for receiving the sample collection tool when the sample collection tool is sufficiently inserted through the inlet port, and wherein the binary tree of medicines distributed within the agar portion is arranged radially around an axis of the sample insertion area.

In some aspects, the techniques described herein relate to a device, wherein the sample receptacle further includes a seal covering the inlet port.

In some aspects, the techniques described herein relate to a method of performing analysis of a sample, the method including: opening a sealed sample collection device, wherein the sample collection device includes: a sample collection tool configured to collect and hold a sample; and a sample receptacle including: an agar portion, wherein the agar portion includes a binary tree of medicines distributed within the agar portion; and an inlet port configured to receive an end of a sample collection tool, wherein sufficient insertion of the sample collection tool through the inlet port causes the sample to be placed in contact with the agar portion, and wherein sufficient insertion of the sample collection tool through the inlet port causes the inlet port to be sealed thereby preventing outside contaminants from entering the sample receptacle. collecting the sample on the end of the sample collection tool; and sufficiently inserting the end of sample collection tool into the inlet port of the sample receptacle, thereby placing the sample in contact with the agar portion and causing the inlet port to be sealed.

In some aspects, the techniques described herein relate to a method, wherein the agar portion includes a recessed sample insertion area for receiving the sample collection tool when the sample collection tool is sufficiently inserted through the inlet port.

In some aspects, the techniques described herein relate to a method, wherein the agar portion further includes a recessed sample insertion area for receiving the sample collection tool when the sample collection tool is sufficiently inserted through the inlet port, wherein a seal covers the recessed sample insertion area, and wherein the method further includes: sufficiently inserting the end of sample collection tool into the inlet port of the sample receptacle, thereby breaking the seal.

In some aspects, the techniques described herein relate to a method, wherein the sample collection tool includes a tube, swab, or strip.

In some aspects, the techniques described herein relate to a method, wherein the binary tree of medicines distributed within the agar portion includes a plurality of adjacent regions within the agar portion containing one or more antifungals or antibacterials.

In some aspects, the techniques described herein relate to a method, wherein the agar portion further includes a recessed sample insertion area for receiving the sample collection tool when the sample collection tool is sufficiently inserted through the inlet port, and wherein the binary tree of medicines distributed within the agar portion is arranged radially around an axis of the sample insertion area.

In some aspects, the techniques described herein relate to a method, wherein the sample receptacle further includes a seal covering the inlet port.

### BRIEF DESCRIPTION OF THE DRAWINGS

These and other features, aspects, and advantages of the disclosure are described with reference to drawings of certain embodiments, which are intended to illustrate, but not to limit, the present disclosure. It is to be understood that the accompanying drawings, which are incorporated in and constitute a part of this specification, are for the purpose of illustrating concepts disclosed herein and may not be to scale.

FIGS. 1A-1B show side cutaway views of an example cartridge system, in accordance with embodiments of the present application.

FIG. 2A shows a side cutaway view of an example cartridge system with a schematic representation of a binary tree of medicines and biological toxins that may be distributed in agar, in accordance with embodiments of the present application.

FIG. 2B shows a top-down view of a schematic representation of a binary tree of medicines and biological toxins that may be distributed in agar, in accordance with embodiments of the present application.

FIG. 3 shows an example of a differential diagnosis that may be provided by the cartridge system.

FIG. 4 presents a block diagram illustrating an embodiment of a computer hardware system configured to run software for implementing one or more embodiments of the systems and methods disclosed herein.

### DETAILED DESCRIPTION

Although several embodiments, examples, and illustrations are disclosed below, it will be understood by those of ordinary skill in the art that the inventions described herein extend beyond the specifically disclosed embodiments, examples, and illustrations and includes other uses of the inventions and obvious modifications and equivalents thereof. Embodiments of the inventions are described with reference to the accompanying figures, wherein like numerals refer to like elements throughout. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

As mentioned briefly above and as will now be explained in more detail below with reference to the example embodiments provided in the figures, this application describes systems, methods, and graphical user interfaces for remotely proctored, at-home drug testing. Embodiments of the inventions described herein can comprise several novel features, and no single feature is solely responsible for the desirable attributes or is essential to practicing the inventions described.

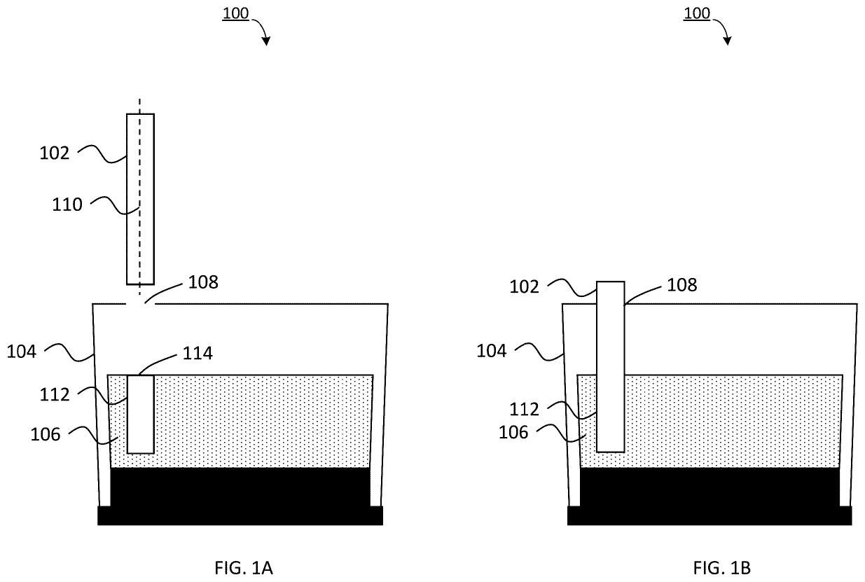

FIGS. 1A and 1B depict an example of the cartridge system 100. The system 100 includes a sample collection tool 102 used for collecting the sample and a sample receptacle 104 configured to receive the sample collection tool 102. The sample collection tool may be a tube, swab, strip, or other medium for collecting and holding a sample (e.g., at a distal end of the sample collection tool). The sample receptacle 104 may include agar 106 (e.g., an agar sheet, pool, or section) disposed therein such that when the sample collection tool is snapped into the sample receptacle through an inlet port 108 (e.g., when the sample collection tool is sufficiently inserted), the sample is placed in contact with the agar 106 and the inlet port 108 is sealed (e.g., sealing off the interior of the sample receptacle 104 to prevent outside contaminants from entering). The size and shape of the inlet port 108 may correspond to (e.g., may be approximately the same as) the size and shape of a cross-section of the sample receptacle taken along a longitudinal axis 110. The agar 106 may also include a recessed area 112 that is configured to receive the sample collection tool 102. The recessed area may align longitudinally with the inlet port 108 and may have a similar cross-sectional size and shape to the sample collection tool 102. In some embodiments, a seal 114 may cover the agar and the recessed area 112 to prevent contamination of the agar. In such configurations, the act of snapping the sample collection tool 102 into the sample receptacle 104 may break the seal 114 and allow the tool 102 and sample therein to contact the agar 106. The seal may be a latex material configured to maintain a sterile agar, though other seal materials may be used as a matter of design choice.

Inside the agar 106, a binary tree of medicines and biological toxins (e.g. different types of antifungal and/or antibacterial treatments, etc.) may be distributed.

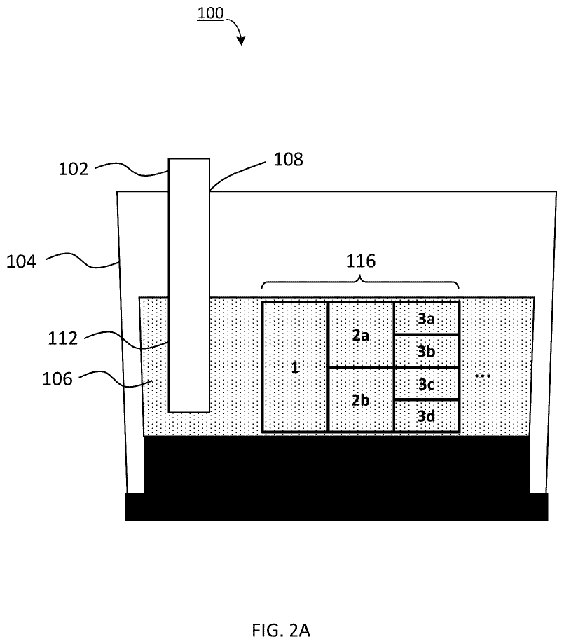

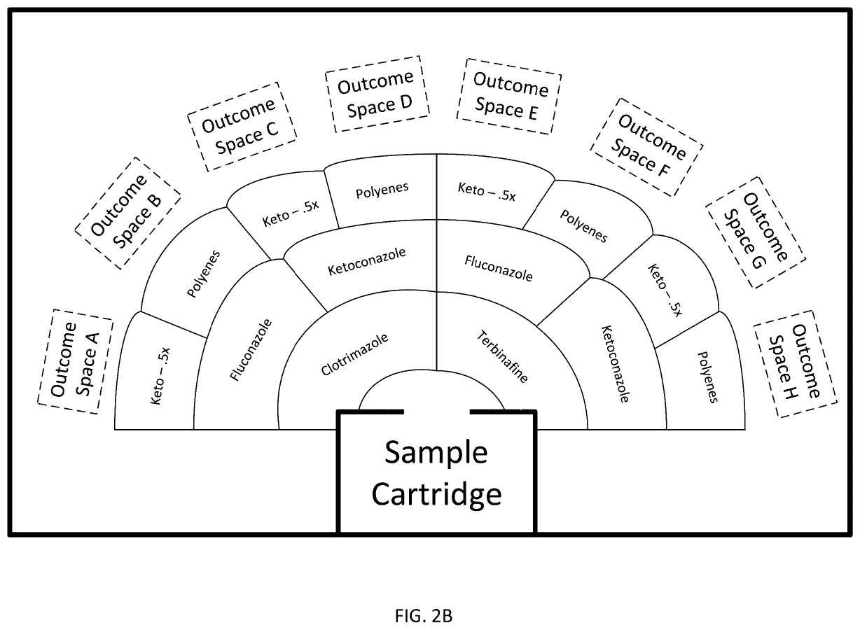

FIG. 2A shows a side cutaway view of an example cartridge system 100 with a schematic representation of a binary tree of medicines and biological toxins that may be distributed in agar 106. Boxes 1, 2A, 2B, 3A, 3B, 3C, 3D, each represent a different type or concentration of antifungal or antibacterial medication. The binary tree of medicines may be located in region 116 of the agar 106 next to the sample insertion area. In some embodiments, the binary tree may include different preparations, concentrations, or strengths of one or more antifungals and/or antibacterials (e.g., located in different adjacent spaces, regions, sections, etc.). The column of medication located furthest from the sample insertion area may be referred to as the outcome space. In the example of FIG. 2A, the column containing medicines 3A, 3B, 3C, 3D may be the outcome space.

Over time, the mold or bacteria from the collected sample will grow or attempt to grow throughout the agar, through the binary tree, toward the outcome space. The outcome spaces act as a type of “score” for each set of the possible antifungal and/or antibacterial treatments contained within the binary tree. The relevant parameters for this score may include the time it took for the sample to reach the outcome space region (or, if the sample did not reach the outcome space, the location along the binary tree where the sample growth stopped), the density at which the outcome space is filled with mold and/or bacteria, or other parameters. The scores can be created, read, or interpreted by the user and/or by a proctor, an artificial intelligence (“AI”) AI system, or other computer vision-based scoring system or algorithm. In some embodiments, the user may be guided through the testing experience and results interpretation by a proctor and/or an AI system. Graphics may be included with the test kit or may be printed on or fixed to the sample receptacle that assist with reading results (e.g., a silkscreen print on the front of the agar panel outlining each box or column within the binary tree). The graphics may assist a user, proctor, a camera (e.g., a smartphone camera) with a CV algorithm and the like in reading test results. In some embodiments, the test kit may also include a dedicated piece of hardware (e.g. a custom case for the agar panel with LEDs on one side and photodiodes on the other) that may be used in results interpretation.

The score will inform medical practitioners about the nature of the sample via differential diagnosis and can be used to create the least invasive treatment procedure for the patient.

FIG. 2B shows a top down view of a schematic representation of an example binary tree of medicines and biological toxins that may be distributed in agar (e.g., within a cartridge system), similar to the binary tree shown in FIG. 2A but the columns of medication are shown to extend outwards radially from the axis of the sample insertion area. In some embodiments, the binary tree may include different preparations, concentrations, or strengths of one or more antifungals and/or antibacterials. The column of medication located furthest from the sample insertion area may be referred to as the outcome space (e.g., outcome spaces A-H shown in the figure). For instance, in FIG. 2B, outcome space B may correspond to the growth path through Clotrimazole, Fluconazole, and Polyenes; any scoring of outcome space B may be associated with that particular group of treatments.

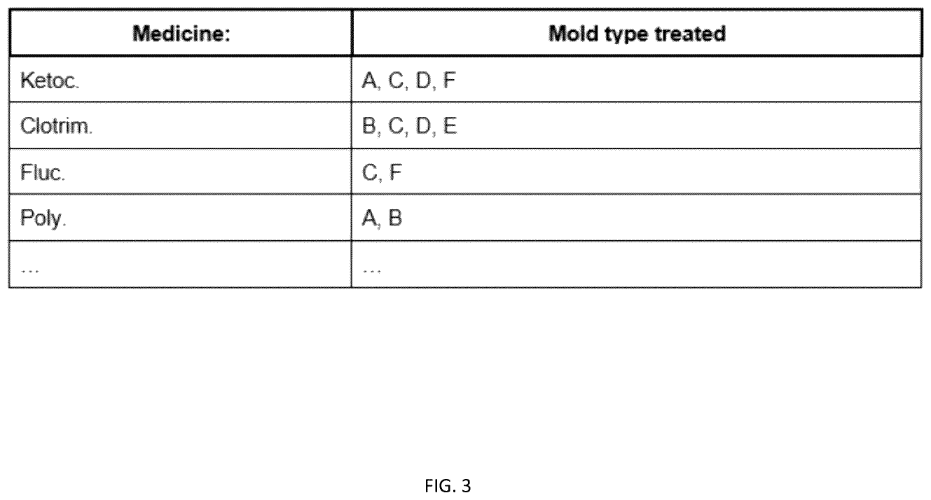

FIG. 3 shows an example of a differential diagnosis that may be provided by the cartridge system 100. In a completed test, the scores of each outcome space are determined (e.g., on a scale based on amount of sample growth in the outcome space or on a binary rating of whether or not sample growth is present in the outcome space, etc.). The results in this example indicate that the paths passing through Ketoc. and Clotrim. were slowed and did not reach large population sizes. The paths passing through Fluc. showed more growth than the paths without them.

From this, it can be deduced that the sample likely includes mold type D—the shared mold type between the highly effective medicines are mold types C and D, and the sample growth through Fluc. suggests that the sample is unlikely to contain mold type C. Additionally, the test provides information about how the identified mold type responds to combinations of medications and different concentrations (e.g., Ketoc. 0.5× strength was highly effective when mixed with Terb., but not without Terb.). With this information, a customized polytherapy from a compounding pharmacy may be created to treat the patient (or the patient's environment) using a highly specific approach that minimizes harm.

An example method for testing may begin with a user opening a sealed sample collection tool. A sample may then be collected in/on the open sample collection tool 102 (e.g., patient spits into a cup and dips a swab into the saliva, a swab is taken of the environment/air vents/mold area, ground/well water is poured into the tube, etc.). The user may then insert the sample collection tool through a port in the sample receptacle. In some embodiments, the port may be sealed and may require the user to remove or break the seal before inserting the sample collection tool. The user may then further insert the sample collection tool into the agar sheet. This may include breaking a seal covering a recessed portion of the agar sheet that is configured to receive the sample collection tool.

The tool may include geometry such that it clicks or locks into sample receptacle and/or the sheet of agar, thereby indicating to the user that the insertion action is complete. The locking or clicking may also create a seal between the cartridge and the receptacle and/or a seal between the cartridge and the agar which blocks off air/liquids from the outside world entering the sample receptacle 104. The locking or clicking may also be associated with breaking the seal on the agar sheet, which allows the sample to be exposed to the agar. Other methods for testing may include all, some, or more of the steps listed above.

### Computer Systems

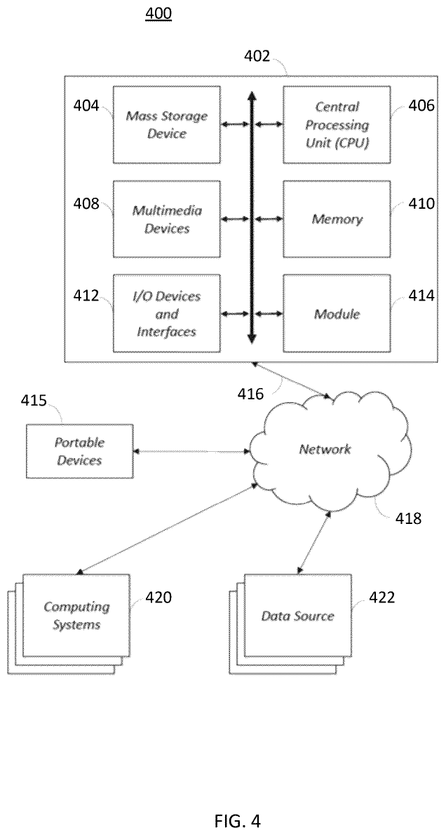

FIG. 4 is a block diagram depicting an embodiment of a computer hardware system configured to run software for implementing the systems, methods, and devices disclosed herein (e.g., the computer vision/AI approach for scoring outcome spaces). The example computer system 402 is in communication with one or more computing systems 420 and/or one or more data sources 422 via one or more networks 418. While FIG. 4 illustrates an embodiment of a computing system 402, it is recognized that the functionality provided for in the components and modules of computer system 402 may be combined into fewer components and modules, or further separated into additional components and modules.

The computer system 402 can comprise a module 414 that carries out the functions, methods, acts, and/or processes described herein. The module 414 is executed on the computer system 402 by a central processing unit 406 discussed further below.

In general, the word “module,” as used herein, refers to logic embodied in hardware or firmware or to a collection of software instructions, having entry and exit points. Modules are written in a program language, such as JAVA, C or C++, PYTHON or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 402 includes one or more processing units (CPU) 406, which may comprise a microprocessor. The computer system 402 further includes a physical memory 410, such as random-access memory (RAM) for temporary storage of information, a read only memory (ROM) for permanent storage of information, and a mass storage device 404, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 3202 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 402 includes one or more input/output (I/O) devices and interfaces 412, such as a keyboard, mouse, touch pad, and printer. The I/O devices and interfaces 412 can include one or more display devices, such as a monitor, which allows the visual presentation of data to a user. More particularly, a display device provides for the presentation of GUIs as application software data, and multi-media presentations, for example. The I/O devices and interfaces 412 can also provide a communications interface to various external devices. The computer system 402 may comprise one or more multi-media devices 408, such as speakers, video cards, graphics accelerators, and microphones, for example.

The computer system 402 may run on a variety of computing devices, such as a server, a Windows server, a Structure Query Language server, a Unix Server, a personal computer, a laptop computer, and so forth. In other embodiments, the computer system 402 may run on a cluster computer system, a mainframe computer system and/or other computing system suitable for controlling and/or communicating with large databases, performing high volume transaction processing, and generating reports from large databases. The computing system 402 is generally controlled and coordinated by an operating system software, such as z/OS, Windows, Linux, UNIX, BSD, SunOS, Solaris, MacOS, or other compatible operating systems, including proprietary operating systems. Operating systems control and schedule computer processes for execution, perform memory management, provide file system, networking, and I/O services, and provide a user interface, such as a graphical user interface (GUI), among other things.

The computer system 402 illustrated in FIG. 4 is coupled to a network 418, such as a LAN, WAN, or the Internet via a communication link 416 (wired, wireless, or a combination thereof). Network 418 communicates with various computing devices and/or other electronic devices. Network 418 is communicating with one or more computing systems 420 and one or more data sources 422. The module 414 may access or may be accessed by computing systems 420 and/or data sources 422 through a web-enabled user access point. Connections may be a direct physical connection, a virtual connection, and other connection type. The web-enabled user access point may comprise a browser module that uses text, graphics, audio, video, and other media to present data and to allow interaction with data via the network 418.

Access to the module 414 of the computer system 402 by computing systems 420 and/or by data sources 422 may be through a web-enabled user access point such as the computing systems' 420 or data source's 422 personal computer, cellular phone, smartphone, laptop, tablet computer, e-reader device, audio player, or another device capable of connecting to the network 418. Such a device may have a browser module that is implemented as a module that uses text, graphics, audio, video, and other media to present data and to allow interaction with data via the network 418.

The output module may be implemented as a combination of an all-points addressable display such as a cathode ray tube (CRT), a liquid crystal display (LCD), a plasma display, or other types and/or combinations of displays. The output module may be implemented to communicate with input devices 412 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 402 may comprise a physical or logical connection established between a remote microprocessor and a mainframe host computer for the express purpose of uploading, downloading, or viewing interactive data and databases on-line in real time. The remote microprocessor may be operated by an entity operating the computer system 402, including the client server systems or the main server system, an/or may be operated by one or more of the data sources 422 and/or one or more of the computing systems 420. In some embodiments, terminal emulation software may be used on the microprocessor for participating in the micro-mainframe link.

In some embodiments, computing systems 420 who are internal to an entity operating the computer system 402 may access the module 414 internally as an application or process run by the CPU 406.

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 402 may include one or more internal and/or external data sources (for example, data sources 422). In some embodiments, one or more of the data repositories and the data sources described above may be implemented using a relational database, such as DB2, Sybase, Oracle, CodeBase, and Microsoft® SQL Server as well as other types of databases such as a flat-file database, an entity relationship database, and object-oriented database, and/or a record-based database.

The computer system 402 may also access one or more databases 422. The databases 422 may be stored in a database or data repository. The computer system 402 may access the one or more databases 422 through a network 418 or may directly access the database or data repository through I/O devices and interfaces 412. The data repository storing the one or more databases 422 may reside within the computer system 402.

### Additional Embodiments

In the foregoing specification, the systems and processes have been described with reference to specific embodiments thereof. It will, however, be evident that various modifications and changes may be made thereto without departing from the broader spirit and scope of the embodiments disclosed herein. The specification and drawings are, accordingly, to be regarded in an illustrative rather than restrictive sense.

Indeed, although the systems and processes have been disclosed in the context of certain embodiments and examples, it will be understood by those skilled in the art that the various embodiments of the systems and processes extend beyond the specifically disclosed embodiments to other alternative embodiments and/or uses of the systems and processes and obvious modifications and equivalents thereof. In addition, while several variations of the embodiments of the systems and processes have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes of the embodiments of the disclosed systems and processes. Any methods disclosed herein need not be performed in the order recited. Thus, it is intended that the scope of the systems and processes herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

It will also be appreciated that conditional language used herein, such as, among others, “can,” “could,” “might,” “may,” “for example,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open- ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the embodiments are not to be limited to the particular forms or methods disclosed, but, to the contrary, the embodiments are to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (for example, as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (for example, as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein but are to be accorded the widest scope consistent with this disclosure, the principles and the novel features disclosed herein.

We claim:

1. A device for performing analysis of a sample, the device comprising:
a sample collection tool configured to collect and hold a sample; and
a sample receptacle comprising:
an agar portion, wherein the agar portion comprises a binary tree of medicines distributed within the agar portion; and
an inlet port configured to receive an end of a sample collection tool, wherein sufficient insertion of the sample collection tool through the inlet port causes the sample to be placed in contact with the agar portion, and wherein sufficient insertion of the sample collection tool through the inlet port causes the inlet port to be sealed thereby preventing outside contaminants from entering the sample receptacle.

2. The device of claim 1, wherein the agar portion comprises a recessed sample insertion area for receiving the sample collection tool when the sample collection tool is sufficiently inserted through the inlet port.

3. The device of claim 1, wherein the agar portion further comprises a recessed sample insertion area for receiving the sample collection tool when the sample collection tool is sufficiently inserted through the inlet port, wherein a seal covers the recessed sample insertion area, and wherein the seal is broken when the sample collection tool is sufficiently inserted through the inlet port thereby placing the sample in contact with the agar portion.

4. The device of claim 1, wherein the sample collection tool comprises a tube, swab, or strip.

5. The device of claim 1, wherein the binary tree of medicines distributed within the agar portion comprises a plurality of adjacent regions within the agar portion containing one or more antifungals or antibacterials.

6. The device of claim 1, wherein the agar portion further comprises a recessed sample insertion area for receiving the sample collection tool when the sample collection tool is sufficiently inserted through the inlet port, and wherein the binary tree of medicines distributed within the agar portion is arranged radially around an axis of the sample insertion area.

7. The device of claim 1, wherein the sample receptacle further comprises a seal covering the inlet port.

8. A method of performing analysis of a sample, the method comprising:
opening a sealed sample collection device, wherein the sample collection device comprises:
a sample collection tool configured to collect and hold a sample; and
a sample receptacle comprising:
an agar portion, wherein the agar portion comprises a binary tree of medicines distributed within the agar portion; and
an inlet port configured to receive an end of a sample collection tool, wherein sufficient insertion of the sample collection tool through the inlet port causes the sample to be placed in contact with the agar portion, and wherein sufficient insertion of the sample collection tool through the inlet port causes the inlet port to be sealed thereby preventing outside contaminants from entering the sample receptacle.
collecting the sample on the end of the sample collection tool; and
sufficiently inserting the end of sample collection tool into the inlet port of the sample receptacle, thereby placing the sample in contact with the agar portion and causing the inlet port to be sealed.

9. The method of claim 8, wherein the agar portion comprises a recessed sample insertion area for receiving the sample collection tool when the sample collection tool is sufficiently inserted through the inlet port.

10. The method of claim 8, wherein the agar portion further comprises a recessed sample insertion area for receiving the sample collection tool when the sample collection tool is sufficiently inserted through the inlet port, wherein a seal covers the recessed sample insertion area, and wherein the method further comprises:
sufficiently inserting the end of sample collection tool into the inlet port of the sample receptacle, thereby breaking the seal.

11. The method of claim 8, wherein the sample collection tool comprises a tube, swab, or strip.

12. The method of claim 8, wherein the binary tree of medicines distributed within the agar portion comprises a plurality of adjacent regions within the agar portion containing one or more antifungals or antibacterials.

13. The method of claim 8, wherein the agar portion further comprises a recessed sample insertion area for receiving the sample collection tool when the sample collection tool is sufficiently inserted through the inlet port, and wherein the binary tree of medicines distributed within the agar portion is arranged radially around an axis of the sample insertion area.

14. The method of claim 8, wherein the sample receptacle further comprises a seal covering the inlet port.
